(19) United States
(12) Patent Application Publication
(10) Pub. No.: US 2008/0241282 A1
(43) Pub. Date: Oct. 2, 2008

(54) Sorghum Extract Compositions

(75) Inventors: Mohamed M. Rafi, Highland Park, NJ (US); Thomas G. Hartman, Staten Island, NY (US); Yesu T. Das, Piscataway, NJ (US); Yassaman Shafaie, Springfield, NJ (US); John Hargreaves, Whitehouse Station, NJ (US)

Correspondence Address: FOX ROTHSCHILD LLP;PRINCETON PIKE CORPORATE CENTER, 2000 Market Street, Tenth Floor, Philadelphia, PA, 19103, US

(73) Assignee: RUTGERS, THE STATE UNIVERSITY, New Brunswick, NJ (US)

(21) Appl. No.: 11/766,104

(22) Filed: Jun. 21, 2007

Related U.S. Application Data
(60) Provisional application No. 60/815,376, filed on Jun. 21, 2006.

(51) Int. Cl. A61K 36/00; A23L 1/28; A61P 35/00

(52) U.S. Cl. 424/725; 426/655

(57) ABSTRACT

Methods and compositions for preventing or treating various diseases or conditions in a patient with an alcohol extractable fraction of Sorghum bicolor grain.

### CROSS REFERENCE TO RELATED APPLICATIONS

This application claims priority to U.S. Provisional Application Ser. No. 60/815,376, which was filed on Jun. 21, 2006, the disclosure of which is incorporated herein by reference.

### BACKGROUND OF THE INVENTION

The use of herbal therapy or alternative medicine is becoming an increasingly attractive approach for the treatment of various inflammatory disorders. Anti-inflammatory properties of various phytochemicals are mediated through the inhibition of production of cytokines (IL-1β, TNF-α, IL-6, IL-12, IFN-γ), nitric oxide (NO), prostaglandins and leukotrienes. Antioxidants such as (−)-epigallocatechin-3-gallate (EGCG), resveratrol, and naturally occurring flavonoids including apigenin and kaempferol have been reported to suppress NO production through inhibition of NF-κB.

Nitric oxide has been defined as an endothelium-derived relaxing factor and the endogenous production of nitric oxide has been shown to play essential roles in the regulation of the physiological process as well as in host defense. In innate immunity, excessive production of nitric oxide is active against invasion of parasites and microorganism and the high production of nitric oxide is associated with cytotoxic and cytostatic activities against bacteria and tumor cells. There is also increasing evidence showing the importance of nitric oxide in the modulation of inflammation, and the overproduction of nitric oxide has been found during the progress of many inflammatory diseases such as rheumatoid arthritis and osteoarthritis. The critical role of NO in various pathological conditions has led to the discovery of new therapeutic agents from varied sources.

Nitric oxide (NO) is a short-lived free radical produced from L-arginine in a reaction catalyzed by NO synthase (NOS). It mediates diverse functions by acting on most cells of the body through the interaction with different molecular targets, which can either be activated or inhibited. At least three types of NOS isoforms have been reported. Endothelial NOS (eNOS) and neuronal NOS (nNOS) are constitutively expressed and are Ca²⁺/calmodulin dependent. Whereas, the high-output isoform, inducible NOS (iNOS), is expressed by cytokines such as interferon (IFN) α, β and -γ and interleukin (IL)-1α and -1β, and lipopolysaccharide (LPS)-activated macrophages and endothelial cells following their transcriptional induction and new protein synthesis. Low concentrations of NO produced by iNOS possess beneficial roles in antimicrobial activity of macrophages against pathogens. At the same time excessive production of NO and its derivatives, such as peroxynitrite and nitrogen dioxide, have been suggested to be mutagenic in vivo, provoke the pathogenesis of septic shock and diverse autoimmune disorders. Furthermore, NO and its oxidized forms have also been shown to be carcinogenic. Therefore, suppressing high NO production by inhibiting iNOS expression and/or its activity may be a therapeutic tool for management of NO-related disorders.

Prostaglandins (PG) are important mediators of inflammation that are produced at elevated levels in inflammation. Prostaglandins are not stored within cells and are produced from fatty acid precursors through stimulation. Prostaglandins can be synthesized through the cyclooxygenase pathway and are involved in a wide variety of physiological roles in mammalian systems. Two different isoforms of cyclooxygenase (COX), designated cyclooxygenase-1 (COX-1) and cyclooxygenase-2 (COX-2), have been identified. COX-1 is a constitutive isoform that exists in most tissues. COX-2 is undetectable in most normal tissues, but it is induced by cytokines, growth factors, oncogenes and tumor promoters. COX-2 is responsible for prostanoid production in inflammation and COX-1 is for the pro stanoids involved in homeostasis. Enhanced levels of COX-2 have been found in numerous human inflammatory conditions including osteoarthritis, rheumatoid arthritis, and acute/chronic inflammatory disease. Since the induction of COX-2 is responsible for the production of PGs at the site of inflammation, it is a possible target for therapeutic purposes.

Nuclear factor-κB (NF-κB) is a transcription factor which plays an important role in promoting inflammation and in the regulation of cell proliferation and survival. NF-κB also stimulates the expression of enzymes whose products contribute to the pathogenesis of the inflammatory process, including cyclooxygenase 2 (COX-2), the inducible form of nitric oxide synthase (iNOS) and a variety of pro-inflammatory cytokines. Interestingly, cytokines that are stimulated by NF-κB, such as TNF-α and IL-1β, are also potent NF-κB inducers, thus establishing a positive autoregulatory loop that can amplify the inflammatory response and lead to chronic inflammation. Consistent with its essential role in inflammation, NF-κB is also known to be the target of anti-inflammatory compounds, including non-steroidal anti-inflammatory drugs. The multiple levels of control of NF-κB activity are not surprising considering the number of genes whose expression is regulated by this factor. NF-κB-binding sites have in fact been identified in the promoter region of more than 150 cellular genes.

Peroxisome proliferator-activated receptors (PPARs) are transcription factors belonging to the superfamily of nuclear receptors. The PPAR gamma agonists are major regulators of lipid and glucose metabolism. Thiazolidinediones (TZDs), synthetic ligands of peroxisome proliferator-activated receptor (PPAR)-gamma, are known to decrease hepatic glucose production and increase glycogen synthesis in diabetic animals. Type 2 diabetes develops in the context of both insulin resistance and beta-cell failure. The improved insulin sensitivity may be achieved either by systemic insulin sensitization or by direct action of peroxisome proliferator-activated receptor (PPAR)-gamma on the transcription of genes involved in glucose disposal. The thiazolidinedione rosiglitazone (Avandia) is a peroxisome proliferator-activated receptor-gamma (PPAR-gamma) agonist, that was recently approved by the Food and Drug Administration for treatment of type II diabetes mellitus.

Peroxisome proliferator activator receptor-alpha (PPAR) and gamma ligands such as fenofibrate and rosiglitazone, respectively, have also been shown to have protective effects on the vessel wall after angioplasty. PPAR-gamma agonists inhibit production of monocyte inflammatory cytokines. PPAR-gamma is expressed in breast, prostate, colon epithelium and administration of synthetic PPAR ligands have been shown to inhibit prostate, breast, and colon tumor cell growth. PPAR-gamma ligands are potent inhibitors of angiogenesis in vivo and in vitro. PPAR-gamma agonists are also used to treat pituitary tumors.

Research in the past decades have accumulated enough evidence to show the beneficial effect of free-radical scavengers/antioxidants as anti-mutagenic, anti-inflammatory, anti-atherosclerotic, anti-diabetic, anti-hepatotoxic, anti-ageing and in a variety of neurological disorders. The research for new antioxidative components is becoming, therefore, critically important to improve the pharmacological treatment of these conditions.

### SUMMARY OF THE INVENTION

The present invention is based upon the finding that sorghum extracts, more specifically, C₁-C₆ polar solvent extractable sorghum fractions, have significant anti-inflammatory, anti-cancer, and anti-diabetic activity. Many of the components found in sorghum extracts, specifically the phenolic compounds, have been shown to have properties that make sorghum a very attractive health promoting grain, compared to other grains that either do not contain similar phenolic compounds or have them to a lesser extent. Of these phenolic compounds, certain sorghum varieties have a well-documented amount of condensed tannins. Other cereal grains, such as wheat, rice and maize, do not contain tannins and barley generally contains tannins in a lower amount as compared to sorghum.

The sorghum extracts according to the present invention inhibit transcription factor NF-κB, through which inhibition of COX-2 and iNOS expression is mediated, so that the sorghum extracts of the present invention have anti-inflammatory properties. Accordingly, one embodiment of the present invention includes a method of treating a chronic or transitory inflammatory disease in a mammal or avian by administering an amount of a C₁-C₆ polar solvent extractable fraction of Sorghum bicolor grain effective to reduce lipopolysaccharide (LPS)-induced nuclear factor-kappa B (NF-κB) activation. Another embodiment includes a method of chronic or transitory inflammatory disease symptom prophylaxis by administering to a mammal or avian in need thereof an LPS-induced NF-κB activation inhibiting amount of a C₁-C₆ polar solvent extractable fraction of Sorghum bicolor effective to prevent inflammatory disease symptoms.

The amount of sorghum extract effective for treating an inflammatory disease or preventing inflammatory disease symptoms in a mammal or avian can be readily determined by one of skill in the art. In one embodiment, the amount of sorghum extract is effective to reduce nitric oxide production. In another embodiment, the amount of sorghum extract is effective to reduce inducible nitric oxide synthase inhibitor (iNOS) production. Exemplary chronic inflammatory diseases include—osteoarthritis, rheumatoid arthritis, asthma, celiac disease, inflammatory bowel disease and inflammation-related cancers, such as colon cancer and prostate cancer, and the like. Sorghum is free of gluten and therefore particularly beneficial in the treatment of celiac disease.

The sorghum extracts according to the present invention are anti-thrombotic and are thus cardio-protective and effective in preventing cardiovascular diseases. Accordingly, another embodiment of the present invention is a method of preventing cardiovascular disease by administering to a patient in need thereof a cardioprotective amount of a C₁-C₆ polar solvent extractable fraction of Sorghum bicolor grain

Soghum extracts according to the present invention also inhibit alpha-amylase activity, which is effective in the prevention and treatment of adult-onset diabetes. Therefore, another embodiment of the present invention is a method of preventing or treating adult onset diabetes in a mammal in need thereof by administering to the mammal an alpha-amylase-inhibiting amount of a C₁-C₆ polar solvent extractable fraction of Sorghum bicolor grain effective to reduce blood sugar levels.

Sorghum extracts according to the present invention are also peroxisome proliferator-activated receptor-gamma (PPAR-gamma) agonists, which are effective in the prevention and treatment of adult-onset diabetes and cellular proliferative disorders. Therefore, another embodiment of the present invention is a method of preventing or treating adult-onset diabetes in a mammal in need thereof by administering a PPAR-gamma activating amount of a C₁-C₆ polar solvent extractable fraction of Sorghum bicolor grain effective to reduce blood sugar levels.

Sorghum extracts according to the present invention also up-regulate FOS gene expression and down-regulate transcription factor NF-κB, which is useful for inducing tumor cell apoptosis. Therefore, yet another embodiment of the present invention includes a method of inducing tumor cell apoptosis by administering to a patient in need thereof an amount of a C₁-C₆ polar solvent extractable fraction of Sorghum bicolor grain effective to induce tumor cell apoptosis. In one embodiment, the tumor cell is a breast tumor cell.

Sorghum extracts according to the present invention also activate estrogen response elements in hormone refractive prostate cancer cell lines. Therefore, another embodiment of the present invention includes a method for treating hormone refractory prostate cancer by administering to a patient in need thereof an amount of a C₁-C₆ polar solvent extractable fraction of Sorghum bicolor grain effective to activate estrogen response element (ERE) in prostate cancer cells.

The sorghum extracts are also useful as natural color pigments food s and cosmetics. Therefore, another embodiment of the present invention is a natural food or cosmetic color pigment, which includes a C₁-C₆ polar solvent extractable fraction of Sorghum bicolor grain.

Also provided is a composition for treating a mammal or avian, wherein the composition includes a C₁-C₆ polar solvent extractable fraction of Sorghum bicolor grain and a pharmaceutically acceptable carrier.

### BRIEF DESCRIPTION OF THE DRAWINGS

FIG. 1 displays the results of a cytotoxicity evaluation of sorghum extracts using MTT dye (1 mg/ml in PBS, pH 7.2);

FIG. 2 shows the inhibition of nitric oxide production from RAW 264.7 by Sorghum extracts;

FIG. 3 is an immunoblot for expression of inducible nitric oxide synthase (iNOS) and cyclooxygenase-2 (COX-2);

FIG. 4 shows the inhibitory effect of sorghum on mRNA expression of inducible nitric oxide synthase (iNOS) and cyclooxygenase-2 (COX-2);

FIG. 5 shows the inhibition of LPS induced NF-κB activation by sorghum; the retarded bands are indicated with an arrow;

FIG. 6A. demonstrates the activation of PPAR-gamma by sorghum extracts; the high intensity band (indicated with arrow) shows that PPAR-gamma is activated with sorghum treatment;

FIG. 6B. shows the activation of PPAR-gamma by sorghum extracts in human colon cancer cell lines Caco-2; the high intensity band (indicated with arrow) shows that PPAR-gamma is activated with sorghum treatment;

FIG. 7 shows the activation of ERE by sorghum extracts in human hormone refractory prostate cancer cell lines Du-145; the high intensity band (indicated with arrow) shows that ERE is activated with sorghum treatment;

FIG. 8 demonstrates that sorghum extracts (1 mg/ml) upregulate the expression of c-FOS genes (identified by the Oligo GEArray® Human Breast Cancer Biomarkers Microarray: OHS-402) compared to control breast cancer cell lines MCF-7;

FIG. 9 shows that unprocessed sorghum increases the expression of FOS gene in MCF-7 cells;

FIG. 10 demonstrates the validation of FOS gene expression by Real-Time PCR; sorghum extracts increase the FOS gene expression by 1.26 fold;

FIGS. 11 and 12 show that unprocessed sorghum decreases the expression of 33 genes in MCF-7 cells;

FIG. 13 demonstrates that sorghum extracts (1 mg/ml) down regulate the expression of 22 genes involved in the NF-κB signaling pathways (identified by the Oligo GEArray® Human NF-κB Signaling Pathway Microarray: OHS-025) compared to control breast cancer cell lines MCF-7;

FIGS. 14 and 15 show that sorghum extract decreases the expression of genes (22) involved in NF-κB pathway;

FIG. 16 demonstrates that processed sorghum extract (120° C.) down regulates the expression of COX-2 and iNOS genes (identified by custom made Oligo GEArray®);

FIG. 17 shows that processed sorghum (120° C.) decreases the expression of COX-2 (Ptgs2) and iNOS(NOS2) genes;

FIG. 18 demonstrates that processed (120 and 170° C.) and unprocessed sorghum extracts inhibit nitric oxide activity;

FIG. 19 is a full-scan mass spectrum and structure of salicylic acid (C₇H₆O₃; MW 138) in sorghum extract analyzed by High Performance Liquid Chromatography Mass Spectrometry (HPLC-MS) under positive ion electrospray conditions;

FIG. 20 is a daughter ion mass spectrum and structure of salicylic acid (C₇H₆O₃; MW 138) in sorghum extract analyzed by High Performance Liquid Chromatography-Mass Spectrometry (HPLC-MS) under positive ion electrospray conditions;

FIG. 21 is a daughter ion mass spectrum and structure of salicylic acid (C₇H₆O₃; MW 138) reference standard analyzed by High Performance Liquid Chromatography-Mass Spectrometry (HPLC-MS) under positive ion electrospray conditions; and

FIG. 22 is a mass spectrum and structure of 3-Hydroxybenzoic acid (C₇H₆O₃; MW 138); the structure of its methyl ester (C₈H₈O₃; MW 152) is shown.

### DETAILED DESCRIPTION OF THE INVENTION

The present invention relates to methods and compositions for preventing or treating various diseases or conditions in a patient with a C₁-C₆ polar solvent extractable fraction, and preferably a C₁-C₆ alcohol extractable fraction or C₃-C₆ acetate extractable fraction, of Sorghum bicolor grain (“sorghum extract”). The Sorghum bicolor grain can be processed or unprocessed. Any suitable solvent can be used for preparing the extractable fraction of Sorghum bicolor grain, provided that the fraction components are essentially the same as the components obtained by extraction with a C₁-C₆ polar solvent. Preferred alcohol extractable fractions include butanol or ethanol extractable fractions, and the preferred acetate extractable fraction is an ethyl acetate extractable fraction.

It is possible process the Sorghum bicolor grain by extrusion processing to make cereal products, food products, pet foods, and the like containing the sorghum extracts of the present invention. Sorghum flours mixed with water and extruded at temperatures between 120 and 170 Centigrade by conventional means will form food products similar to other grain products that retain the activity of the extractable fractions of the present invention.

Sorghum (Sorghum bicolor (L.) Moench), originally from Africa, is part of the grass family and is closely related to sugarcane. Sorghum ranks fifth in terms of importance and consumption around the world after wheat, maize, rice, and barley, and is the fifth most important grain in the U.S after corn, cotton, soybeans, and wheat. Approximately half of all the sorghum grown in the world is produced for animal feed, and the other half is produced for human consumption and for other industrial uses, such as ethanol production, biodegradable packaging, and wallboard for the housing industry. Sorghum is a major staple in the developing world, where it provides food for over 300 million people in Africa and India combined. It can be grown successfully in high altitude, high soil toxicity, water abundance or shortage, and in high and low temperature extremes, making it highly attractive to populations living in regions dealing with deteriorating agricultural lands and water shortages.

Sorghum comes in many varieties and colors that vary in nutritive contents and usage potential. In Africa, Sorghum can be found in a variety of beverages and foods including breads, pancakes, dumplings, couscous, porridges (like ‘to’), and beers (like ‘dolo’). Each cultivar has different characteristics and qualities that make it more or less desirable in the many different foods produced from sorghum in Africa and throughout the world.

The United States is the world's largest sorghum producer, and is also currently the world's largest exporter of grain sorghum, where approximately half of the crop is sold abroad as an animal feed. Sorghum grain consumed domestically is primarily used as a feed for livestock. It is also used in ethanol production. Even though sorghum is exported primarily for use as an animal feed, it is vital for human food production and consumption in the developing world.

The grains range from shades of white, red, brown, yellow, purple, to black; however, sorghum is most commonly found in the colors white, bronze and brown. Sorghum grains of different cultivars are a rich source of phenolic compounds. Phenolic compounds are commonly considered secondary metabolites due to the fact that they are not involved directly in any metabolic processes. Some of these compounds act as protective chemicals within plants and humans. The three major groups of phenolic compounds include phenolic acids, flavonoids, and condensed tannins. All sorghum grains contain phenolic compounds in varying concentrations but only the varieties with pigmented testa, or seed coat, produce tannins. The major phenolic acids that have been isolated are Gallic, Protocatechuic, p-Hydroxybenzoic, Vanillic, Caffeic, p-Coumaric, Ferulic and Cinnamic and Benzoic acid derivatives. The major groups of flavonoids found are called anthocyanidins, and the two most prevalent types are luteoforol and apiforols.

Preliminary methods of identification of phenolic compounds found in sorghum based on visible characteristics are partly available. While the many types and colors of sorghum vary in nutritive qualities, it has been shown that pericarp color is not a determining factor in the identification of type or amount of phenolic compounds in sorghum. The darkest colored seed coats tend to have the highest tannin quantities. One variety of sorghum, the tannin or brown sorghum cultivar, has the highest tannin level and, despite the name, can be seen in a variety of pericarp colors, including white, yellow, and red.

Sorghum cultivars of different variety contain an extensive range of vitamins including the vitamins D, E, K, thiamin, riboflavin, niacin, B6, pantothenic acid, and biotin. Sorghum also contains the carotenoids lutein, zeaxanthin, and β-carotene.

Sorghum contains various important vitamins and minerals that are comparable or in excess of maize and barley and is a strong source of dietary fiber. Fiber has been shown to have a significant inverse correlation with coronary heart disease (CHD), and dietary fiber has also been effectively shown to decrease serum cholesterol and LDL cholesterol concentrations. These studies are important for showing fiber's involvement in lowering the risk of CHDs and related conditions. High fiber diets play a role in controlling diabetes and hyperinsulinemia by improving the body's sensitivity to insulin, helping to lower serum insulin concentrations, and improving glycemic control. Fiber rich foods, like sorghum, slow down digestion and absorption, thus retarding the diabetic's glycemic response and providing yet another mode of glycemic control. In addition to fiber, tannins, a phenolic compound found in sorghum, have been shown to increase the glucose uptake in rat adipocytes and they could also play a role in the lowering of blood glucose levels.

As used above, and throughout the description of the invention, the following terms, unless otherwise indicated, shall be understood to have the following meanings:

“Sorghum Extract” means a C₁-C₆ polar solvent extractable fraction, and preferably a C₁-C₆ alcohol extractable fraction or C₃-C₆ acetate extractable fraction, of Sorghum bicolor grain. Sorghum extracts according to the present invention need only correspond to such extractable fractions and are not necessarily produced by solvent extraction. Accordingly, the sorghum extracts can be a component of sorghum flour both before and after extrusion processing.

“Patient” means a mammal including a human.

“Effective amount” means an amount of Sorghum extract effective for producing a desired therapeutic effect.

“Treat” or “treatment” or “treating” mean to lessen, eliminate, inhibit, improve, alter, or prevent a disease, condition, or disorder, for example by administration of a Sorghum extract.

In practice, a composition containing the Sorghum extract may be administered in any variety of suitable forms, for example, orally, by inhalation, topically, parenterally, or rectally. Preferably, the composition is administered orally. More specific routes of administration include intravenous, intramuscular, subcutaneous, intraocular, intrasynovial, colonical, peritoneal, transepithelial including transdermal, ophthalmic, sublingual, buccal, dermal, ocular, nasal inhalation via insufflation, and aerosol.

A composition containing the Sorghum extract may be presented in forms permitting administration by the most suitable route. The invention also relates to administering compositions containing the Sorghum extract which is suitable for use as a medicament in a patient. These compositions may be prepared according to the customary methods, using one or more pharmaceutically acceptable adjuvants or excipients. The adjuvants comprise, inter alia, diluents, sterile aqueous media and the various non-toxic organic solvents. The compositions may be presented in the form of oral dosage forms, or injectable solutions, or suspensions. Preferred dosage forms include tablets, capsules, oily suspensions, aqueous suspensions, lozenges, troches, powders, granules, emulsions, syrups, and elixirs.

The choice of vehicle is generally determined in accordance with the solubility and chemical properties of the product, the particular mode of administration and the provisions to be observed in pharmaceutical practice. When aqueous suspensions are used they may contain emulsifying agents or agents which facilitate suspension. Diluents such as sucrose, ethanol, polyols such as polyethylene glycol, propylene glycol and glycerol, and chloroform or mixtures thereof may also be used. In addition, the Sorghum extract may be incorporated into sustained-release preparations and formulations.

For parenteral administration, emulsions, suspensions or solutions of the compounds according to the invention in vegetable oil, for example sesame oil, groundnut oil or olive oil, or aqueous-organic solutions such as water and propylene glycol, injectable organic esters such as ethyl oleate, as well as sterile aqueous solutions of the pharmaceutically acceptable salts, are used. The injectable forms must be fluid to the extent that it can be easily syringed, and proper fluidity can be maintained, for example, by the use of a coating such as lecithin, by the maintenance of the required particle size in the case of dispersion and by the use of surfactants. Prolonged absorption of the injectable compositions can be brought about by use of agents delaying absorption, for example, aluminum monostearate and gelatin. The solutions of the salts of the products according to the invention are especially useful for administration by intramuscular or subcutaneous injection. Solutions of the Sorghum extract as a free base or pharmacologically acceptable salt can be prepared in water suitably mixed with a surfactant such as hydroxypropyl-cellulose. Dispersions can also be prepared in glycerol, liquid polyethylene glycols, and mixtures thereof and in oils. The aqueous solutions, also comprising solutions of the salts in pure distilled water, may be used for intravenous administration with the proviso that their pH is suitably adjusted, that they are judiciously buffered and rendered isotonic with a sufficient quantity of glucose or sodium chloride and that they are sterilized by heating, irradiation, microfiltration, and/or by various antibacterial and antifungal agents, for example, parabens, chlorobutanol, phenol, sorbic acid, thimerosal, and the like.

Sterile injectable solutions are prepared by incorporating the Sorghum extract in the required amount in the appropriate solvent with various of the other ingredients enumerated above, as required, followed by filtered sterilization. Generally, dispersions are prepared by incorporating the various sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique, which yield a powder of the active ingredient plus any additional desired ingredient from previously sterile-filtered solution thereof.

Topical administration, gels (water or alcohol based), creams or ointments containing the Sorghum extract may be used. The Sorghum extract may be also incorporated in a gel or matrix base for application in a patch, which would allow a controlled release of compound through transdermal barrier.

For administration by inhalation, the Sorghum extract may be dissolved or suspended in a suitable carrier for use in a nebulizer or a suspension or solution aerosol, or may be absorbed or adsorbed onto a suitable solid carrier for use in a dry powder inhaler.

The percentage of Sorghum extract in the compositions used in the present invention may be varied, it being necessary that it should constitute a proportion such that a suitable dosage shall be obtained. Obviously, several unit dosage forms may be administered at about the same time. A dose employed may be determined by a physician or qualified medical professional, and depends upon the desired therapeutic effect, the route of administration and the duration of the treatment, and the condition of the patient. In the adult, the doses are generally from about 0.001 to about 50, preferably about 0.001 to about 5, mg/kg body weight per day by inhalation, from about 0.01 to about 100, preferably 0.1 to 70, more especially 0.5 to 10, mg/kg body weight per day by oral administration, and from about 0.001 to about 10, preferably 0.01 to 10, mg/kg body weight per day by intravenous administration. In each particular case, the doses are determined in accordance with the factors distinctive to the patient to be treated, such as age, weight, general state of health and other characteristics, which can influence the efficacy of the compound according to the invention.

The Sorghum extract used in the invention may be administered as frequently as necessary in order to obtain the desired therapeutic effect. Some patients may respond rapidly to a higher or lower dose and may find much weaker maintenance doses adequate. For other patients, it may be necessary to have long-term treatments at the rate of 1 to 4 doses per day, in accordance with the physiological requirements of each particular patient. Generally, the Sorghum extract may be administered 1 to 4 times per day. Of course, for other patients, it will be necessary to prescribe not more than one or two doses per day.

The following non-limiting examples set forth herein below illustrate certain aspects of the invention.

### EXAMPLES

### Preparation of Sorghum Extracts

Anti-inflammatory properties of butanol and ethanol extracts of raw sorghum (bronze variety) grown in central New Jersey (Michalenko Farms Inc.) were evaluated. Raw extracts were not cytotoxic to mouse macrophage cell lines (RAW 264.7) and using non-cytotoxic doses we have shown the inhibition of nitric oxide production The inhibition of nitric oxide, COX-2, iNOS and mechanism of action are shown below. Mouse macrophage RAW264.7 cells were selected for this study because they are well-established cell lines used for in-vitro model system of inflammation. We have also used prostate cancer cell line Du-145 and breast cancer cell line MCF-7 to show that sorghum extracts are beneficial for cancer prevention/treatment by inducing apoptosis.

### Effect of Sorghum on RAW 264.7 Cell Viability

To rule out any cytotoxic activity of sorghum on RAW 264.7 cells, a standard cell viability assay (MTT assay) was performed with different doses of sorghum (1.0 mg/ml, 0.5 mg/ml, 0.25 mg/ml, and 0.125 mg/ml) with this cell line for 24 hours. Sorghum has almost no cytotoxic effect on RAW 264.7 cells at 0.125 mg/ml to 1.0 mg/ml concentrations. (FIG. 1).

Effect of Sorghum on Lipopolysaccharide (LPS)-Induced Nitric Oxide Production from Mouse Macrophage RAW 264.7 Cells

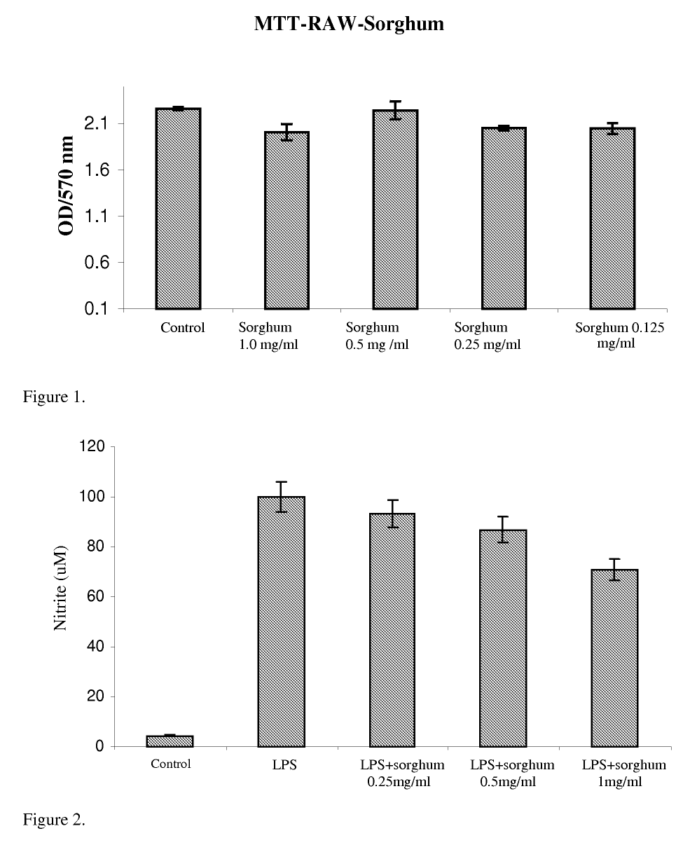

Cell suspensions of RAW 264.7 cells (0.25×106/ml) were prepared. 200 μl/well of the suspension was cultured in a flat bottom microtitre plate for 24 hours. Thereafter, 100 μl of media was replaced with fresh medium containing either LPS (0.5 μg/ml) or LPS and different concentrations of sorghum (1 mg/ml, 0.5 mg/m, and 0.25 mg/ml). The supernatant was harvested after 24 hours. The amount of nitric oxide in the terms of stable product nitrite was quantitated by mixing each well with an equal amount of Griess reagent. Sodium nitrite dissolved in the same medium was used for standard curve formation. FIG. 2 indicates that LPS induced production of nitric oxide is notably inhibited by raw sorghum in a dose dependent manner.

Effect of Sorghum on LPS-Induced iNOS and COX-2 Protein Expression

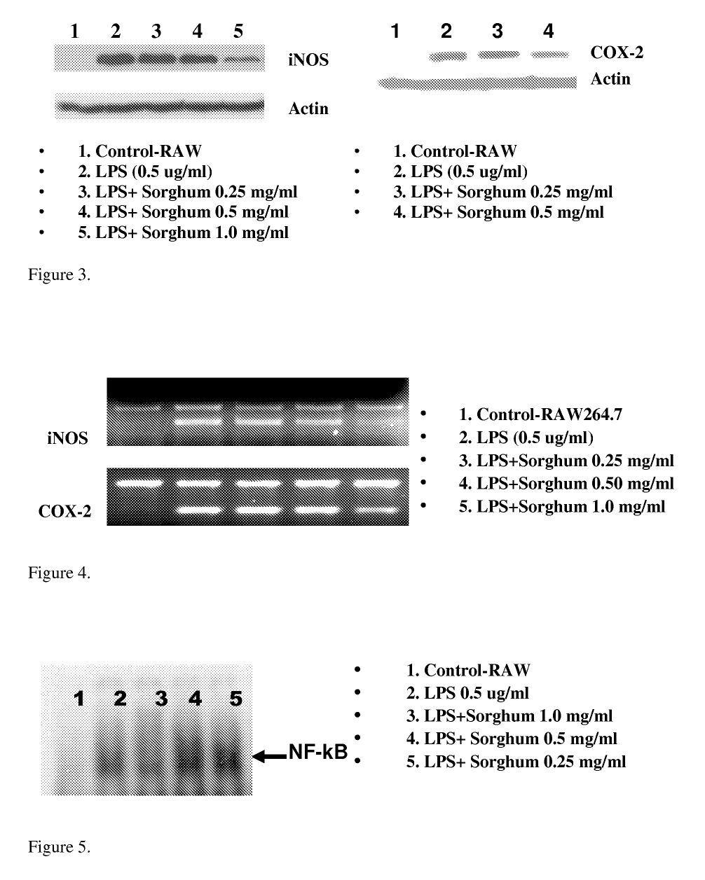

The RAW 264.7 cells were cultured (106 ml) in 6 well plates for 24 hours. The medium was then replaced with fresh medium and different concentrations of sorghum, either alone or in combination with LPS, and further cultured for 24 hours. Thereafter, total cell lysate was prepared by SDS-lamellae sample buffer and boiled for 5 minutes. 20 μl of this cell lysate from each treatment group was resolved on 8-10% SDS-PAGE and transferred to nitrocellulose membrane. The detection of expression iNOS and COX-2 was performed using monoclonal anti iNOS and COX-2. The amount of actin protein was also quantitated as an internal control using monoclonal actin antibody. (FIG. 3).

Effect of Sorghum on LPS-Induced iNOS and COX-2 mRNA Expression

To investigate whether the inhibition of protein expression of iNOS and COX-2 is due to less protein synthesis or due to modulation of post-translational events, the RT-PCR analysis for iNOS and COX-2 gene (Ambion Inc) was performed. The RAW 264.7 cells were cultured (106 ml) in 6 well plates for 24 hours. Then the medium was replaced by fresh medium and different concentrations of raw sorghum extract, either alone or in combination with LPS, and was further cultured for 12 hours. Total RNA was isolated using Tri-reagent (SIGMA, MO, USA), 5 μg of which was reverse transcribed to make cDNA using Oligo-dT and superscript reverse transcriptase (Invitrogen Corp.). Using gene specific primers, 2 μl of cDNA was amplified for 349 base pair (bp) of iNOS, 297 bp of COX-2, and 495 bp of 18S ribosomal RNA by PCR following the manufacturer's instructions. Sorghum at 1 mg/ml inhibited the LPS (0.5 μg/ml) stimulated mRNA expression of iNOS and COX-2 after 12 hours of treatment with RAW 264.7 cells. This inhibition of mRNA correlates to the inhibition of protein expression by sorghum.

Sorghum significantly inhibits the mRNA expression of the iNOS and COX-2 gene at the highest doses. (FIG. 4). This demonstrates anti-inflammatory properties of crude extracts of sorghum.

### Inhibition of LPS-Induced NF-κB Activation by Sorghum

To investigate whether the inhibition of COX-2, NO, and iNOS is mediated through the NF-κB pathway, an electrophoretic mobility shift assay (EMSA) was performed to determine if the NF-κB binding to the promoter regions of the target genes are inhibited by the sorghum extracts. Sorghum extracts at 1.0 mg/ml inhibited the NF-κB binding. The EMSA was performed to analyze NF-κB activation in nuclear protein lysates prepared after 2 hours of sorghum treatment. As shown in FIG. 5, the induction of specific NF-κB DNA binding activity by LPS was inhibited by sorghum. The relative levels of NF-κB DNA binding activity with the treatment of 0.25 mg/ml to 1.0 mg/ml of sorghum were less in comparison to LPS alone. Most optimum inhibition was found at 1.0 mg/ml concentration of sorghum. The specificity of binding was examined by competition with the addition of excess of unlabeled oligonucleotides.

### Activation of PPAR-Gamma Using Sorghum

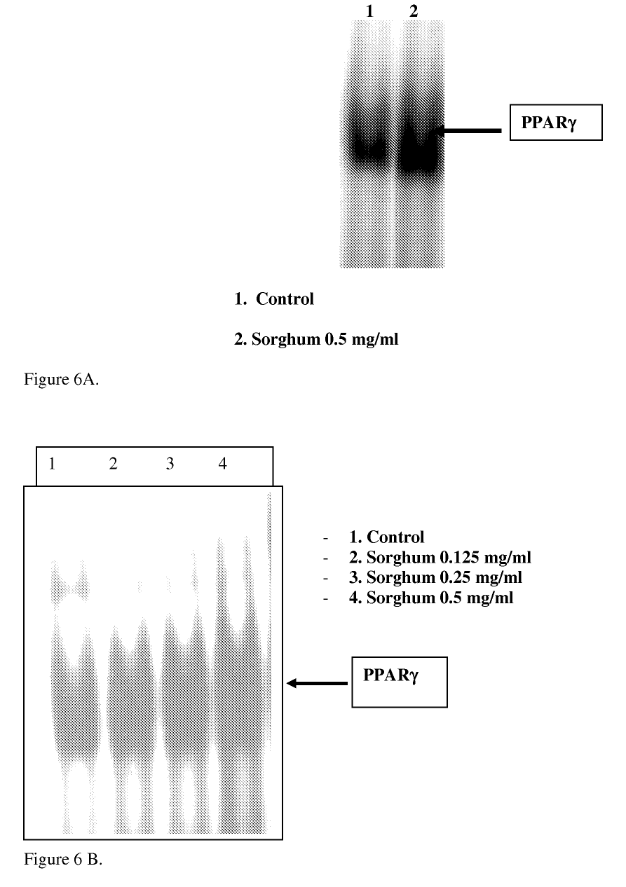

To investigate whether PPAR-gamma expression is activated by sorghum, an EMSA assay was performed to determine if the PPAR-gamma binding to the promoter regions of genes is activated by the sorghum extracts. Sorghum extracts at 1.0 mg/ml activates the PPAR binding in mouse macrophage cell lines (FIG. 6A) and in human colon cancer cell lines (FIG. 6B). EMSA was performed to analyze PPAR activation in nuclear protein lysates prepared after 2 hours of sorghum treatment. As shown in FIG. 6A, the induction of specific PPAR DNA binding activity was increased with sorghum compared with control. The relative levels of PPAR DNA binding activity with the treatment of 0.125 mg/ml to 0.5 mg/ml of sorghum were higher in human colon cancer cells in comparison to control. (FIG. 6B). This data indicates that sorghum activates PPAR gamma binding to the nucleus in mouse macrophages and human colon cancer cell lines.

### Activation of Estrogen Response Elements (ERE) in Prostate Cancer Cell Lines Using Sorghum

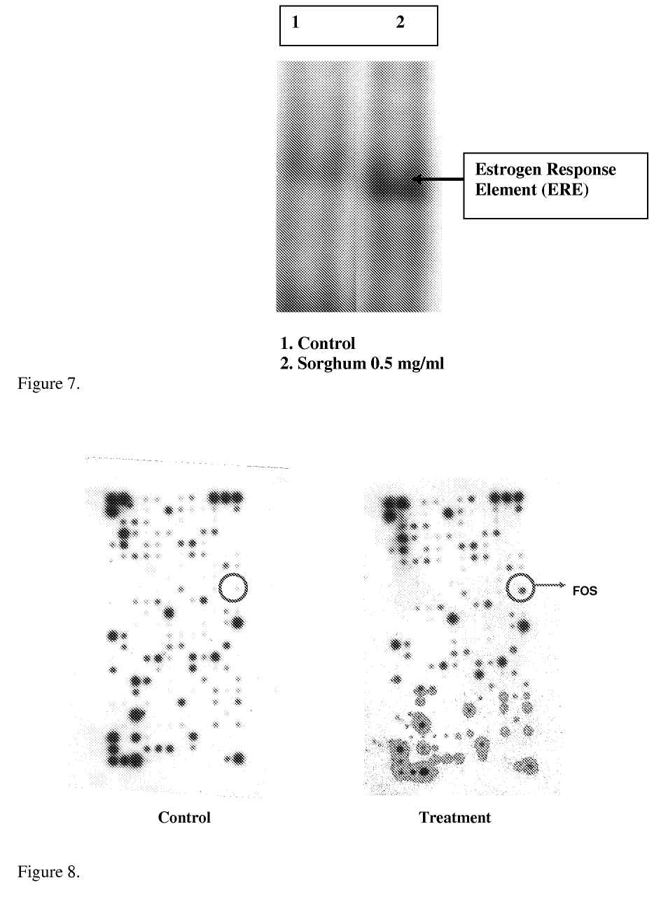

To investigate whether sorghum extracts are estrogenic in nature and activate estrogen response elements (ERE) in prostate cancer cell lines, an EMSA assay was performed to see the ERE activation by the sorghum extracts in DU-145 hormone refractory prostate cancer cell lines. The EMSA was performed to analyze ERE activation in nuclear protein lysates prepared after 2 hours of sorghum treatment. As shown in FIG. 7, the induction of specific ERE DNA binding activity was increased with 0.5 mg/ml sorghum extracts compared to the control. This data indicates that sorghum extracts activate ERE binding to the nucleus.

Sorghum Extracts Upregulate the Expression of c-FOS Genes (Identified by the Oligo GEArray® Human Breast Cancer Biomarkers Microarray OHS-402

To investigate whether sorghum extracts modulate breast cancer biomarker genes, human breast cancer cell lines MCF-7 were treated with sorghum alcohol extracts (1 mg/ml) for 12 hours. The RNA was isolated from the control and treated plates. The isolated RNA was used for the gene expression studies using Oligo-nucleotide arrays containing 264 genes that may serve as diagnosis and/or prognosis markers for breast cancer. These 264 genes are highly associated with breast cancer. The human Oligo GEArray® was purchased from Superarray Bioscience Corporation (Frederick, Md.). The experiments were carried out according to the manufacturer's protocols. From the array analysis, it was determined that c-FOS genes are upregulated with sorghum treatment. (FIG. 8). FOS is a transcription factor which dimerizes with proteins of the JUN family to activate the transcription factor AP-1. In breast cancer cells and clinical specimens the expression of FOS genes has been shown as down regulated. The sorghum extracts upregulate c-FOS genes, thereby inducing apoptosis in breast cancer cells. In general, the FOS proteins have been implicated as regulators of cell proliferation, differentiation, and transformation. In some cases, expression of the FOS gene has also been associated with apoptotic cell death.

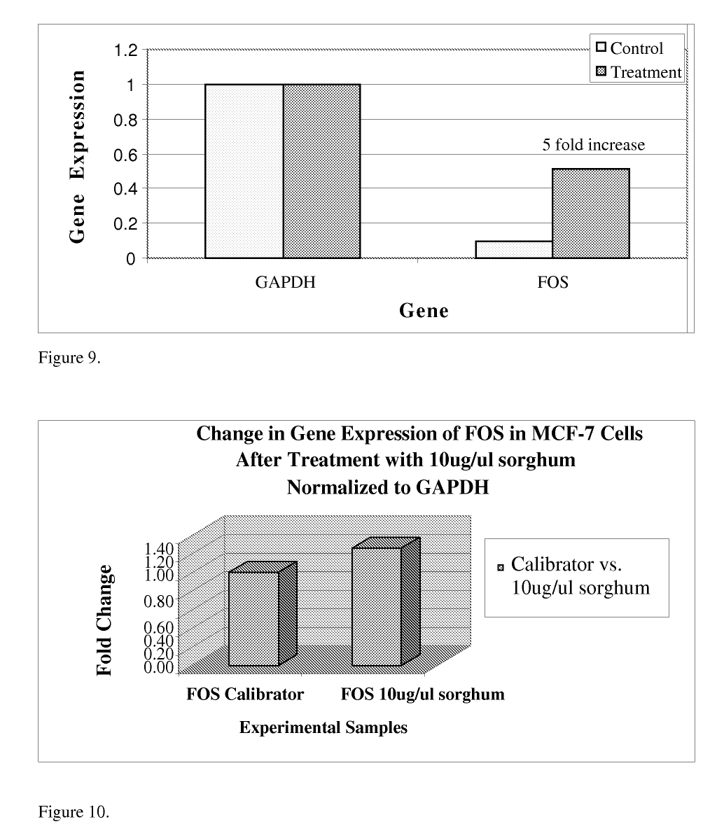

The change in the expression of FOS gene was found to be 5 fold in microarray (FIG. 9) and this was further validated using Real-Time PCR and it was found to be 1.26 fold compared to control (FIG. 10).

### Unprocessed Sorghum Decreases the Expression of 33 Human Breast Cancer Biomarker Genes in MCF-7 Cell Lines (Identified by the Oligo GEArray® Human Breast Cancer Biomarkers Microarray: OHS-402).

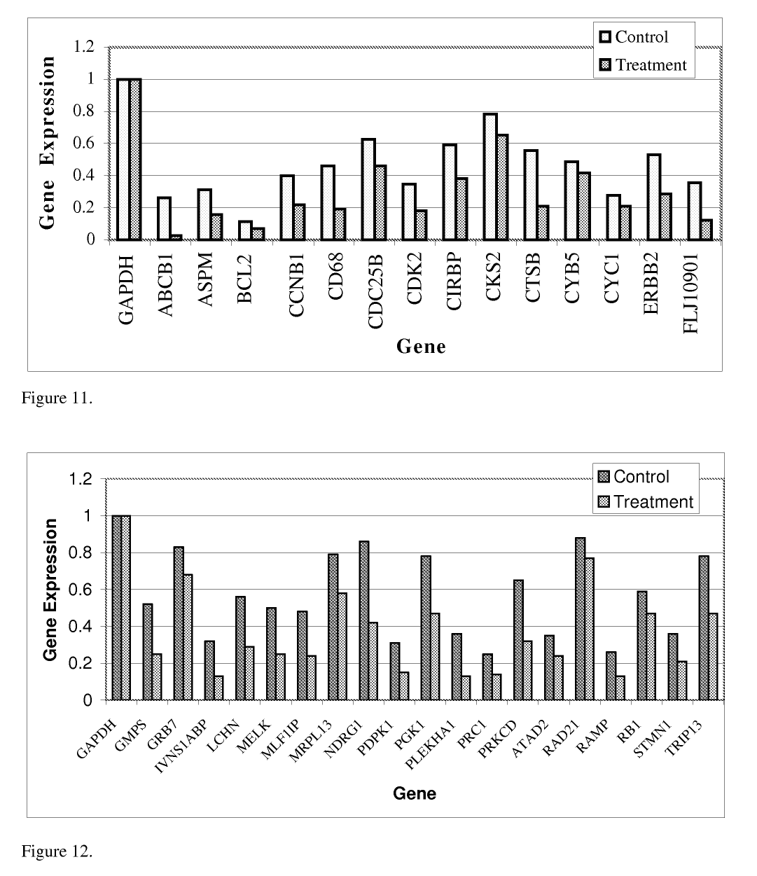

To investigate whether sorghum extracts down regulate the expression of human breast cancer biomarker genes, human breast cancer cell lines MCF-7 were treated with sorghum alcohol extracts (1 mg/ml) for 12 hours. The RNA was isolated from control and treated plates. The isolated RNA was used for the gene expression studies using Oligo-nucleotide Arrays containing 264 genes related to human breast cancer. The genes down regulated are shown in FIGS. 11 and 12.

Sorghum Extracts (1 mg/ml) Down Regulate the Expression of 22 Human NF-κB Pathway Genes (Identified by the Oligo GEArray® Human NF-κB Signaling Pathway Microarray: OHS-025).

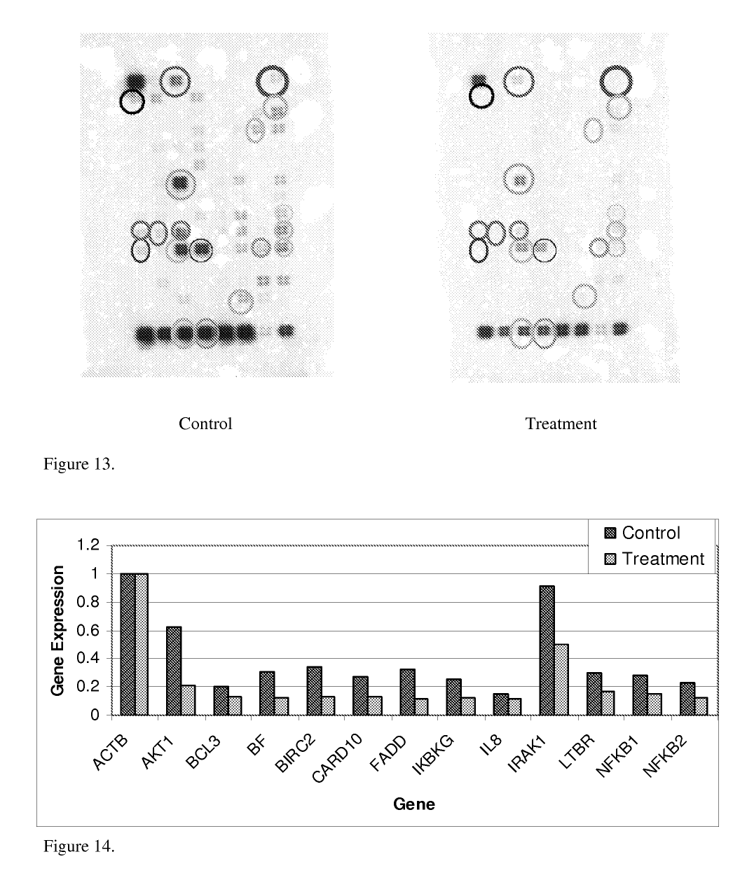

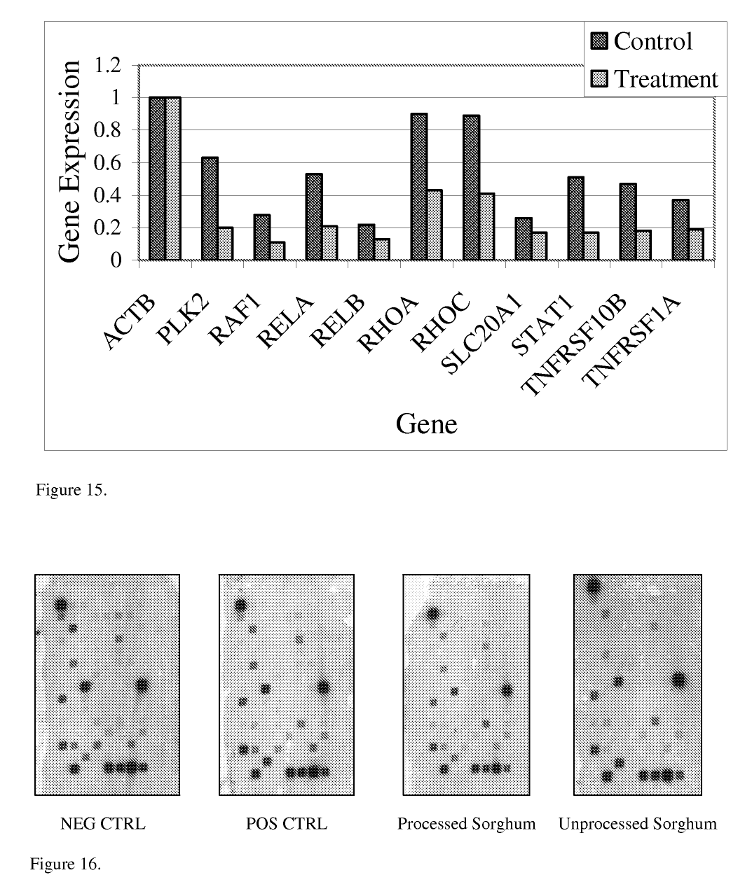

To investigate whether sorghum extracts down regulate the expression of human NF-κB pathway genes, human breast cancer cell lines MCF-7 were treated with sorghum alcohol extracts (1 mg/ml) for 12 hours. The RNA was isolated from control and treated plates. The isolated RNA was used for the gene expression studies using Oligo-nucleotide Arrays containing 113 genes related to NF-κB mediated signal transduction pathways. The human NF-κB Oligo GEArray® was purchased from Superarray Bioscience Corporation (Frederick, Md.). The experiments were carried out according to manufacturer's protocols. From the array analysis, it was determined that 22 genes (AKT1, BCL3, BF, BIRCC2, CARD 10, FADD, IKBKG, IL8, IRAK1, LTBR, NFKB1, NFKB2, PLK2, RAF, RELA, RELB, RHOA, RHOC, SLC2OA1,STAT1, TNFRSF10B and TNFRSF1A) were down regulated with 1 mg/ml sorghum treatment (FIG. 13). The array includes genes that encode members of the Rel, NF-κB, and IkB families, NF-κB-responsive genes, extracellular ligands and receptors that activate the pathway, and kinases and transcription factors that propagate the signal. NF-κB-mediated signal transduction has been implicated in the regulation of viral replication, autoimmune diseases, the inflammatory response, tumorigenesis and apoptosis. With the OHS-025 Oligo GEArray® Human NF-κB Signaling Pathway, it was demonstrated that 22 genes are intensely down regulated with sorghum treatment (FIGS. 14 and 15).

Processed Sorghum Extract (120° C.) Down Regulates the Expression of COX-2 and iNOS Genes Identified by Custom Made Oligo GEArray

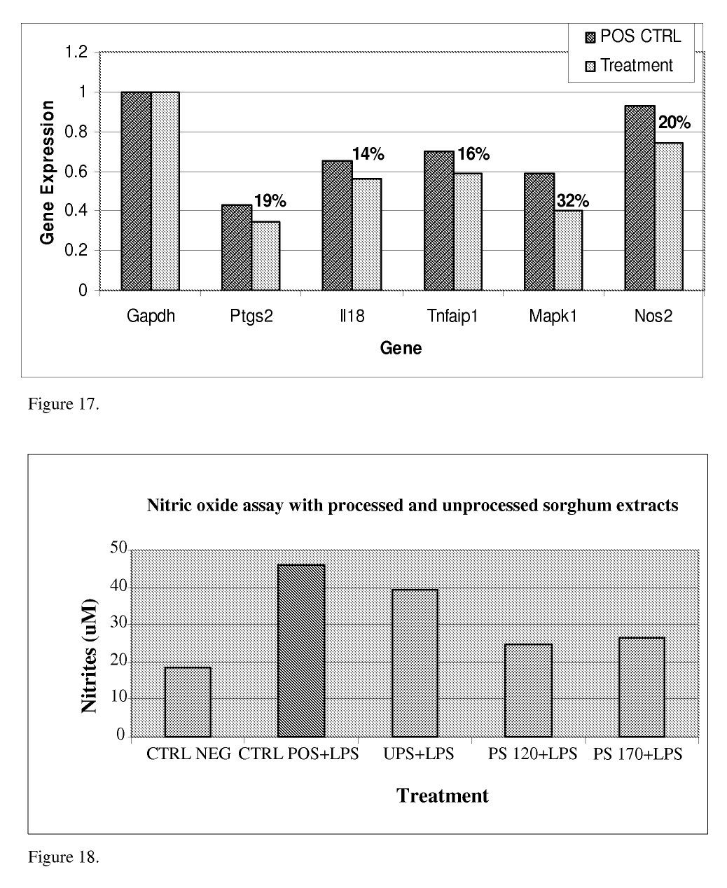

To investigate whether processed sorghum extracts (120° C.) retain anti-inflammatory activities and down regulate the expression of inflammatory genes, mouse macrophage cell lines were treated with processed sorghum alcohol extracts (1 mg/ml) for 12 hours. The RNA was isolated from negative, positive control and treated plates. The isolated RNA was used for the gene expression studies using Oligo-nucleotide Arrays containing 120 genes (FIG. 16) and the data analysis shows that COX-2 and iNOS genes were down regulated by 19-20% as compared to control (FIG. 17).

### Processed Sorghum at High Temperatures (120 and 170° C.) Retains Anti-Inflammatory Activities by Inhibiting Nitric Oxide Production in Mouse Macrophage Cell Lines

Anti-inflammatory properties of ethanol extracts of processed sorghum at two temperatures (120 and 170° C.) were evaluated. It was determined that processed extracts are not cytotoxic to mouse macrophage cell lines (RAW 264.7). Inhibition of nitric oxide production using mouse macrophage cells was demonstrated with non-cytotoxic doses of processed extracts. (FIG. 18). The inhibition of nitric oxide shows that the anti-inflammatory properties are not lost during processing.

### α-Amylase Inhibition by Sorghum

The assay method is based on the principle that the hydrolysis of 2-chloro-4-nitrophenyl-α-D-maltotrioside, catalyzed by α-Amylase, yields 2-chloro-4-nirophenol that is quantitatively measured by its absorbance at 405 nm. Its formation is directly proportional to the α-Amylase activity.

Ten grams of unprocessed sorghum powder was mixed with 15 mL of aqueous buffer (Phosphate Buffer Saline, 10 mM, pH 7.4, PBS), sonicated for 5 minutes in an ultrsonic water bath, and shaken in an Orbit Shaker at 250 rpm for 30 min. It was then centrifuged at 2,000 rpm at 10° C. for 2 hr, and filtered through 0.45 p PTFE membrane filter. The resulting clear solution was used for the enzyme assay.

A 96-well plate was used to conduct the reaction, and a Bio-Rad Microplate Reader (Model No. 680) was used to obtain the Optical Density (OD₄₀₅).

The assay was carried out with α-Amylase (1,4-α-D-Glucan-glucanohydrolase; E.C. 3.2.1.1), equivalent of 10 μL human saliva, and 100 μL of sample extract (100 μL PBS for control). A total volume of 150 μL of reaction mixture was used per well. The plate was incubated for 30 minute over a warm plate (˜30° C.), covering the multiwell plate loosely with a plastic lid. 40 μL of the substrate (2-Chloro-4-nitrophenyl-α-D-maltotrioside) solution was added and allowed to react for 3 minutes. Optical Density was then measured at 405 nm.

The control reaction (without Sorghum extract) yielded a mean OD value of 1.985, while the treated reaction (with Sorghum extract) yielded a mean OD value of 0.624. Thus, the Sorghum extract exhibited 68.56% inhibition of α-Amylase activity.

### Identification of Anti-Inflammatory Molecules in Alcohol Extracts of Processed and Unprocessed Sorghum

### Sample Preparation

The sorghum grain (bronze variety) was ground in a laboratory mill to make sorghum flour. The flour was extruded at 120° C. to simulate expanded and unexpanded products, using a Brabender 1-inch single extruder with a 3:1 compression ratio. The presence of anti-inflammatory compounds in this processed sorghum was investigated, using mass spectrometric techniques (GC-MS and LC-MS/MS).

### Identification of Salicylic Acid in Processed Sorghum Using High Performance Liquid Chromatography-Mass Spectrometry (HPLC-MS)

Processed sorghum (120° C.) was extracted with ethanol (20 g/30 mL) by sonication for 5 minutes in an ultrasonic water bath, and shaking in an Orbit Shaker at 250 rpm for 30 min. It was then centrifuged at 2,000 rpm at 10° C. for 2 hours, and filtered through 0.45 p PTFE membrane filter. The resulting clear solution was used for analysis.

A Finnigan MAT Triple Stage Quadrupole Mass Spectrometer (Model TSQ 700) was used under Positive Ion Electrospray conditions. For the full scan spectra, masses of 50-650 were scanned at a scan time of 1.2 seconds. For the daughter ion spectra, argon gas was employed at a collision cell pressure of 1.2 mTorr and collision energy of 25 V. Instrument control and data acquisition were performed by a Digital Alpha Station 200 (4/166) and Finnigan ICIS software (Version 8.3) for Digital UNIX Operating System (OSF/1) (Version 4.0) (ICL Version 7.5).

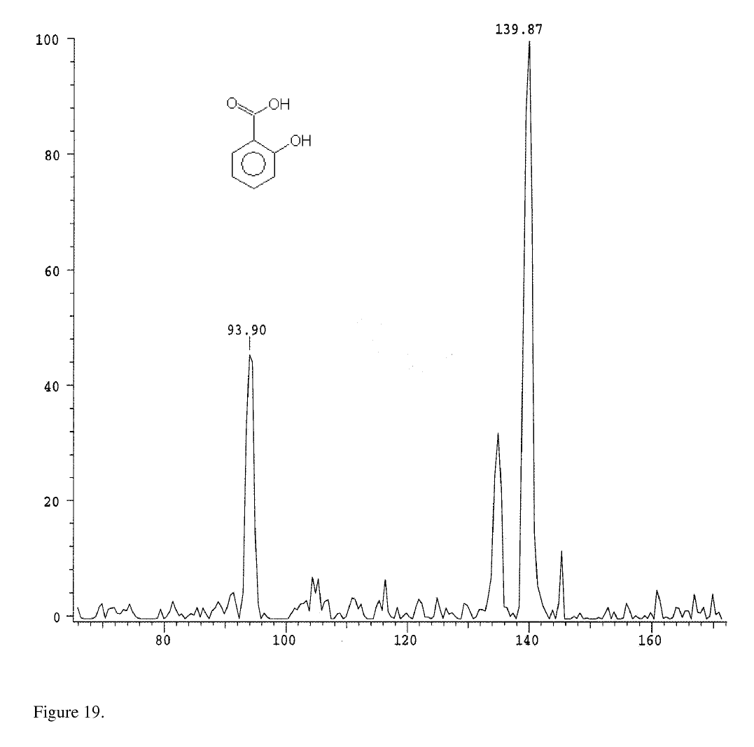

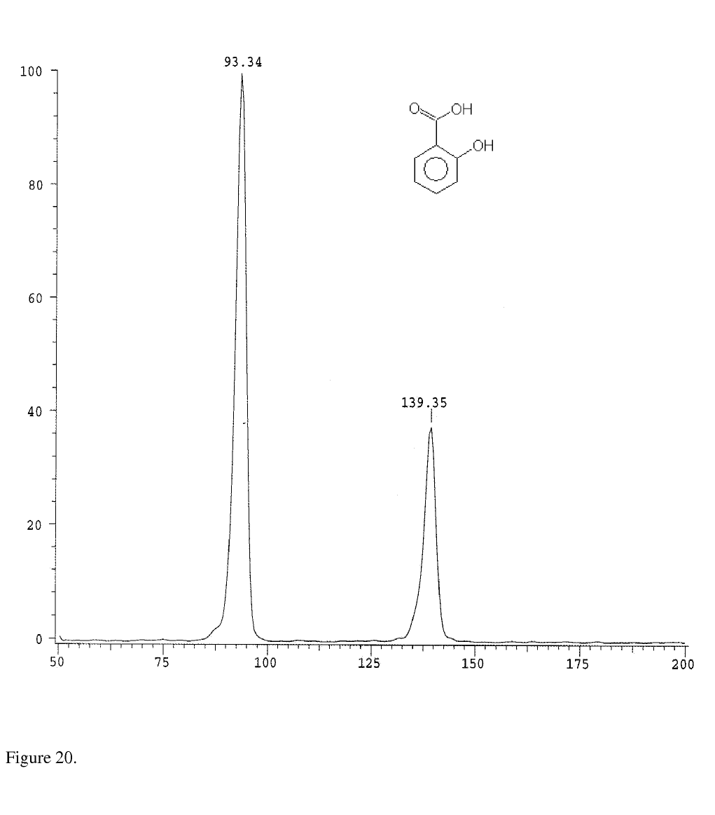

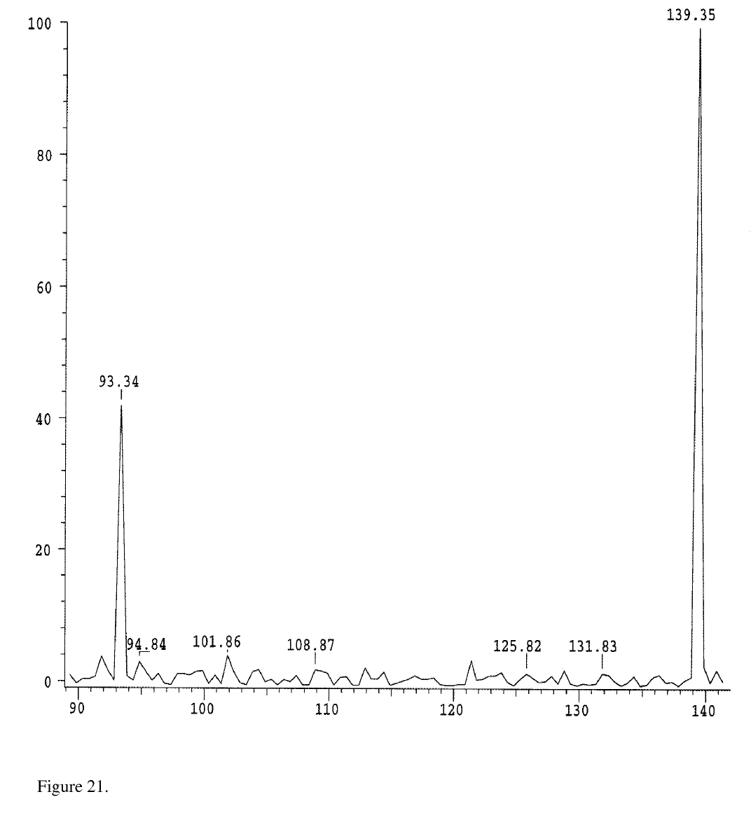

The sample was first analyzed under full-scan conditions (50-650 amu), in which a protonated molecular ion [M+H⁺] of 139 was observed. (FIG. 19). This molecular ion, together with its fragment ion of 93, was suggestive of Salicylic acid (C₇H₆O₃; MW 138). In order to confirm this finding, the sample was then analyzed under MS/MS (daughter ion mode) for the molecular ion [M+H⁺] of 139. The spectrum (FIG. 20) confirmed the earlier observation of the structure being that of salicylic acid. Additional confirmation was also obtained with an authentic reference standard of Salicylic acid, by comparing its daughter ion spectrum (FIG. 21) with that of Sorghum extract (FIG. 20).

Identification of an Analog of Salicylic Acid (meta-Salicylic acid) by Gas Chromatography-Mass Spectrometry (GC-MS)

Processed sorghum (120° C.) was extracted with ethanol (20 g/30 mL) by sonication for 5 min in an ultrasonic water bath, and shaking in an Orbit Shaker at 250 rpm for 30 minutes. It was then centrifuged at 2,000 rpm at 10° C. for 2 hr, and filtered through 0.45 p PTFE membrane filter. The resulting clear solution was methylated with diazomethane, and used for injection into GC-MS.

Analysis was performed under the following GC-MS conditions:

Gas Chromatograph: Varian 3400. Column: RTX-5, 30 meters, 0.32 mm diameter, 0.25, film thickness. Injector temperature: 260° C. Injection volume: 1.0 μL. Temperature Program: 50° C. for 3 minutes, then to 320° C. at 10° C./min.

Mass Spectrometer Finnigan MAT 8230, with SS300 Data System. Interface Line Temperature: 320° C. Ion Source Temperature: 250° C. Filament Emission Current 0.5 mA. Mass Range: 35-650.

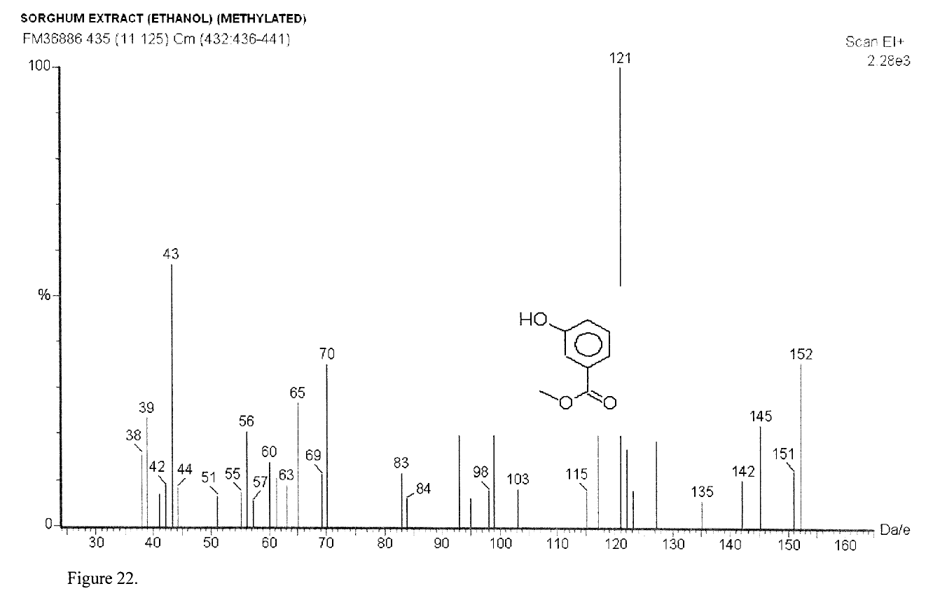

The chromatogram was carefully examined for the presence of organic molecules that have structural relationship with anti-inflammatory molecules. A hydroxy derivative of Benzoic acid (meta-salicylic acid or 3-hydroxy benzoic acid) was detected in the sample. Its spectrum and structure are shown below. (FIG. 22).

### Identification of Major Phytochemicals in the Alcohol Extract of Processed and Unprocessed Sorghum by GC-MS

The alcohol extract contained a mixture of long chain C₆-C₂₀ fatty acids, primarily Palmitic, Oleic and Stearic acids, waxes (myristyl oleate), vitamin E (alpha-tocopherol), mono and diglycerides, phytosterols- such as Ergot-5-en-3-ol, Sigmasterol, beta-Sitosterol, Stigmast-4-en-3-one, and Triterpene alcohol ester-Lupenyl acetate. The other phytochemicals found were: Butylene glycol, trans-2-Heptanal, Heptyl alcohol, Ethyl octanoate, Glycerol, 2,4-Decadienal, Myristic alcohol, Eugenol methyl ether, Ethyl laurate, Ethyl myristate, Methyl palmitate, 6,10,14-Trimethyl-2-pentadecanone, Ethyl pentadecanoate and Nonanedioic acid.

The foregoing examples and description of the preferred embodiments should be taken as illustrating, rather than as limiting the present invention as defined by the claims. As will be readily appreciated, numerous variations and combinations of the features set forth above can be utilized without departing from the present invention as set forth in the claims. Such variations are not regarded as a departure from the spirit and script of the invention, and all such variations are intended to be included within the scope of the following claims.

What is claimed is:

1. A method of treating a chronic or transitory inflammatory disease in a mammal or avian in need thereof comprising administering an amount of a C₁-C₆ polar solvent extractable fraction of Sorghum bicolor grain effective to reduce lipopolysaccharide (LPS)-induced nuclear factor-kappa B (NF-κB) activation.

2. The method of claim 1, wherein said amount is effective to reduce nitric oxide production.

3. The method of claim 1, wherein said amount is effective to reduce inducible nitric oxide synthase inhibitor (iNOS) production.

4. The method of claim 1, wherein said amount is effective to reduce cyclooxygenase-2 production.

5. The method of claim 1, wherein said Sorghum bicolor grain comprises a processed grain.

6. The method of claim 1, wherein said extractable fraction is a C₁-C₆ alcohol extractable fraction or a C₃-C₆ acetate extractable fraction.

7. The method of claim 6, wherein said extractable fraction is an ethanol extractable fraction, a butanol extractable fraction, or an ethyl acetate extractable fraction.

8. The method of claim 1, wherein said chronic inflammatory disease is selected from the group consisting of osteoarthritis, rheumatoid arthritis, celiac disease, inflammatory bowel disease, inflammation-related cancers and asthma.

9. A method of chronic or transitory inflammatory disease symptom prophylaxis comprising administering to a mammal or avian in need thereof an LPS-induced NF-κB activation inhibiting amount of a C₁-C₆ polar solvent extractable fraction of Sorghum bicolor effective to prevent inflammatory disease symptoms.

10. The method of claim 9, wherein said amount is effective to reduce nitric oxide production.

11. The method of claim 9, wherein said amount is effective to reduce inducible nitric oxide synthase inhibitor (iNOS) production.

12. The method of claim 9, wherein said amount is effective to reduce cyclooxygenase-2 production.

13. The method of claim 9, wherein said Sorghum bicolor grain comprises a processed grain.

14. The method of claim 9, wherein said extractable fraction is a C₁-C₆ alcohol extractable fraction or a C₃-C₆ acetate extractable fraction.

15. The method of claim 14, wherein said extractable fraction is an ethanol extractable fraction, a butanol extractable fraction, or an ethyl acetate extractable fraction.

16. A method of preventing or treating adult onset diabetes in a mammal in need thereof comprising administering to said mammal an amount of a C₁-C₆ polar solvent extractable fraction of Sorghum bicolor grain effective to reduce blood sugar levels.

17. The method of claim 16, wherein said Sorghum bicolor grain comprises a processed grain.

18. The method of claim 16, wherein said extractable fraction is a C₁-C₆ alcohol extractable fraction or a C₃-C₆ acetate extractable fraction.

19. The method of claim 18, wherein said extractable fraction is an ethanol extractable fraction, a butanol extractable fraction, or an ethyl acetate extractable fraction.

20. A method of inducing tumor cell apoptosis comprising administering to a patient in need thereof an amount of a C₁-C₆ polar solvent extractable fraction of Sorghum bicolor grain effective to induce tumor cell apoptosis.

21. The method of claim 20, wherein said tumor cell is a breast tumor cell.

22. The method of claim 20, wherein said Sorghum bicolor grain comprises a processed grain.

23. The method of claim 20, wherein said extractable fraction is a C₁-C₆ alcohol extractable fraction or a C₃-C₆ acetate extractable fraction.

24. The method of claim 23, wherein said extractable fraction is an ethanol extractable fraction, a butanol extractable fraction, or an ethyl acetate extractable fraction.

25. A method for treating hormone refractory prostate cancer comprising administering to a patient in need thereof an amount of a C₁-C₆ polar solvent extractable fraction of Sorghum bicolor grain effective to activate estrogen response element (ERE) in prostate cancer cells.

26. The method of claim 25, wherein said Sorghum bicolor grain comprises a processed grain.

27. The method of claim 25, wherein said extractable fraction is a C₁-C₆ alcohol extractable fraction or a C₃-C₆ acetate extractable fraction.

28. The method of claim 27, wherein said extractable fraction is an ethanol extractable fraction, a butanol extractable fraction, or an ethyl acetate extractable fraction.

29. A composition for treating a mammal or avian comprising a C₁-C₆ polar solvent extractable fraction of Sorghum bicolor grain and a pharmaceutically acceptable carrier.

30. The composition of claim 29, wherein said Sorghum bicolor grain comprises a processed grain.

31. The composition of claim 29, wherein said extractable fraction is a C₁-C₆ alcohol extractable fraction or a C₃-C₆ acetate extractable fraction.

32. The method of claim 31, wherein said extractable fraction is an ethanol extractable fraction, a butanol extractable fraction, or an ethyl acetate extractable fraction.

33. The composition of claim 29, wherein said composition is in the form of a tablet, a capsule, an oily suspension, an aqueous suspension, a lozenge, a troche, a powder, a granule, an emulsion, a syrup, or an elixir.

34. A natural food or cosmetic color pigment comprising a C₁-C₆ polar solvent extractable fraction of Sorghum bicolor grain.

35. The pigment of claim 34, wherein said Sorghum bicolor grain comprises a processed grain.

36. The pigment of claim 34, wherein said extractable fraction is a C₁-C₆ alcohol extractable fraction or a C₃-C₆ acetate extractable fraction.

37. The method of claim 36, wherein said extractable fraction is an ethanol extractable fraction, a butanol extractable fraction, or an ethyl acetate extractable fraction.

38. A food product comprising sorghum flour processed by extrusion at a temperature between 120 and 170 Centigrade.
